(19) United States
(12) Patent Application Publication
(10) Pub. No.: US 2008/0058880 A1
(43) Pub. Date: Mar. 6, 2008

(54) SYSTEM AND METHOD FOR PACING

(76) Inventors: Jaeho Kim, Redmond, WA (US); Joseph M. Bocek, Seattle, WA (US); Anthony Harrington, Woodinville, WA (US)

Correspondence Address: SCHWEGMAN, LUNDBERG & WOESSNER, P.A., P.O. BOX 2938, MINNEAPOLIS, MN, 55402, US

(21) Appl. No.: 11/469,595

(22) Filed: Sep. 1, 2006

(51) Int. Cl. A61N 1/00

(52) U.S. Cl. 607/9

(57) ABSTRACT

In an embodiment, an implantable medical device senses the heart rate of a patient by analyzing a cardiac signal of the patient. The device identifies an increase in the patient's sensed heart rate and determines whether noise in the cardiac signal exceeds a certain specified amount. Upon an increase in the sensed heart rate and a finding of noise in the cardiac signal, pacing is invoked in the patient at a normal rate or at a rate that is greater than an estimated intrinsic rate for that patient. The device continues to pace until the device determines that the noise in the cardiac signal has subsided.

### TECHNICAL FIELD

Various embodiments relate to the field of medical devices, and in an embodiment, but not by way of limitation, to implanted medical devices that deliver pacing pulses to a patient.

### BACKGROUND

The heart is an electro-mechanical system that is the center of a person's circulatory system. The heart includes four chambers—the right atrium (RA), the right ventricle (RV), the left atrium (LA), and the left ventricle (LV)—and with these four chambers, the heart performs two major pumping functions. The left portions of the heart, including the LA and the LV, draw oxygenated blood from the lungs and pump it to tissues throughout the body to provide the tissues with their metabolic needs for oxygen. The right portions of the heart, including the RA and the RV, draw deoxygenated blood from a body's tissues and pump it to the lungs where the blood gets re-oxygenated. The efficiency of the pumping functions, which is indicative of whether the heart is normal and healthy, can be measured by the hemodynamic performance of the heart. The hemodynamic performance of the heart can be measured by various parameters that relate to, for example, intracardiac blood pressures and cardiac output.

In a normal heart, the sinoatrial node, the heart's natural pacer, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions indicated by normal hemodynamic performance. A blocked or otherwise abnormal electrical conduction and/or deteriorated myocardial tissue can cause non-synchronous contraction of the heart, resulting in poor hemodynamic performance, including a diminished blood supply to the heart or the rest of the body. Congestive heart failure can occur when the heart fails to pump enough blood to meet the body's metabolic needs.

There are numerous patient conditions that may require the use of a cardiac management device. For example, a bradyarrhythmia patient is a person whose intrinsic heart beat is, at least at certain times, below a level necessary to meet hemodynamic needs. If a bradyarrhythmia patient is constantly below the level needed to sustain hemodynamic functions, that patient is pacer dependent, and the pacer must constantly supply pacing pulses or other therapy to the patient's heart. If a patient experiences sporadic episodes of bradyarrhythmia, then the pacer may supply the therapy on an as needed basis. A tachyarrhythmia patient is one whose heart rate is at times accelerated, which can also diminish hemodynamic function. For a tachyarrhythmia patient, a pacer/defibrillator may deliver anti-tachyarrhythmia pacing or counter shock therapy to interrupt the tachyarrhythmia. In other patients, the atria or ventricles may contract out of synchrony. For example, when a left ventricle becomes enlarged, it may not contract synchronously with the right ventricle, reducing cardiac output. Cardiac resynchronization therapy (CRT) pulses may be delivered to such a patient, such as to bring the ventricles back into synchrony.

Any implantable medical device, just like any electronic device in general, may be adversely affected by noise in its environment. For example, if there is noise in a pacer environment, the pacer may interpret that noise as a heart beat, and then erroneously conclude that the patient does not need pacing therapy. This undesirable inhibition of pacing caused by the presence of noise may lead to a patient succumbing to syncope. Current devices employ systems and methods to handle and respond to such noise, but such current devices and systems suffer from several shortcomings. Consequently, the medical device art is in need of a different method and system to handle noise in medical device environments.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various examples discussed in the present document.

FIG. 1 illustrates a block diagram of an example embodiment of an implantable medical device.

FIGS. 2A-2D illustrate flow charts of example embodiments of processes to deliver pacing pulses to a heart.

FIG. 3 illustrates an example embodiment of an implanted pacer in communication with an external device via a telemetry system.

FIG. 4 illustrates a graph of ventricular contraction intervals.

FIG. 5 illustrates a trace of a cardiac signal and a noise window.

### SUMMARY

In an embodiment, an implantable medical device senses the heart rate of a patient by analyzing a cardiac signal of the patient. The device identifies an increase in the patient's sensed heart rate and determines whether noise in the cardiac signal exceeds a certain specified amount. Upon an increase in the sensed heart rate and a finding of noise in the cardiac signal, pacing is invoked in the patient at a normal rate or at a rate that is greater than an estimated intrinsic rate for that patient. The device continues to pace until the device determines that the noise in the cardiac signal has subsided.

This summary is intended to provide an overview of the subject matter of the present disclosure. It is not intended to provide an exclusive or exhaustive explanation of the disclosure. The detailed description is included to provide further information about the subject matter of the present disclosure.

### DETAILED DESCRIPTION

In the following detailed description, reference is made to the accompanying drawings which form a part hereof, and in which is shown by way of illustration specific embodiments in which the invention may be practiced. These embodiments, which are also referred to as examples, are discussed in sufficient detail to enable those skilled in the art to practice the invention, and it is to be understood that the embodiments may be combined, or that other embodiments may be utilized and that structural, logical and electrical changes may be made without departing from the scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment.

The system and method described herein provide an improved pacing therapy to medical device implant patients. The system and method may be particularly beneficial to patients with demand pacers. Specifically, the present inventors have recognized that in current systems and methods, pacing pulses may be inhibited when there is noise present in the medical device environment. The present disclosure therefore addresses this and other issues.

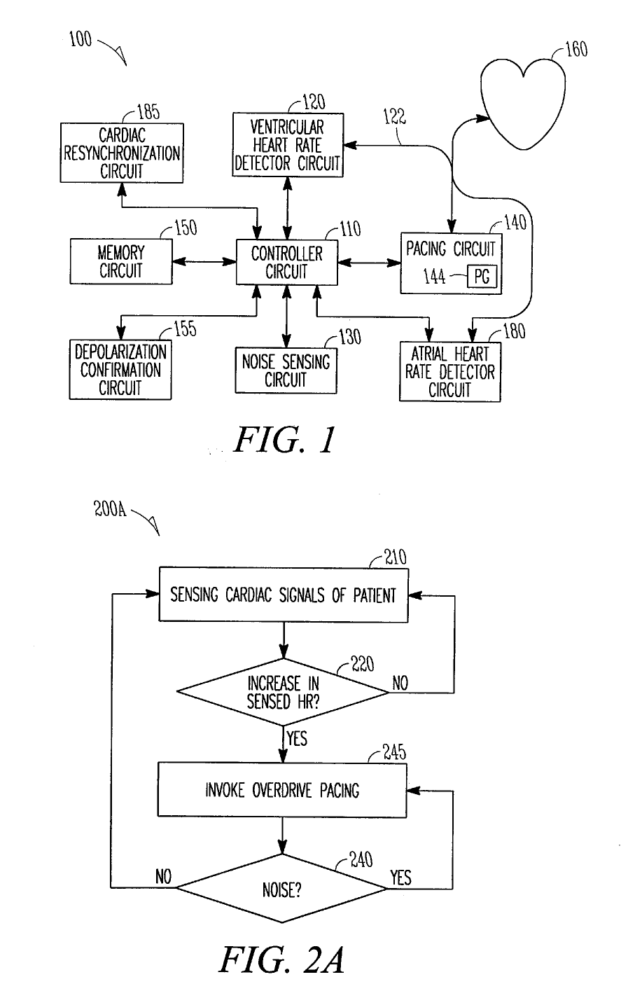

FIG. 1 illustrates an example embodiment of an implantable medical device. The device 100 includes a controller circuit 110, a ventricular heart rate detector circuit 120, a noise sensing circuit 130, a pacing circuit 140 including a pulse generator (PG) 144, a memory circuit 150, a depolarization confirmation circuit 155, an atrial heart rate detector circuit 180, and a cardiac resynchronization therapy circuit 185. The ventricular heart rate detector circuit 120 and the atrial heart rate detector circuit 180 can be connected to one or more leads 122 that may be implanted in association with the wall of the cardiac tissue of a heart 160. Similarly, the pacing circuit 140 can be connected to the one or more leads 122.

In an example, the heart rate detector circuit 120 senses and monitors the heart rate of the heart 160. The sensed heart rate may include intrinsic events, paced events, or a combination of intrinsic and paced events. The heart rate detector circuit 120 processes the signals received from the heart, identifies the depolarization portions of the signal that are indicative of a heart beat, and determines the estimated heart rate from these signals. However, noise in the system may cause the heart rate circuit 120 to erroneously identify such noise as a depolarization indicative of a heart beat. If this noise is erroneously interpreted as a heart beat, pacing therapy may not be delivered when it is needed.

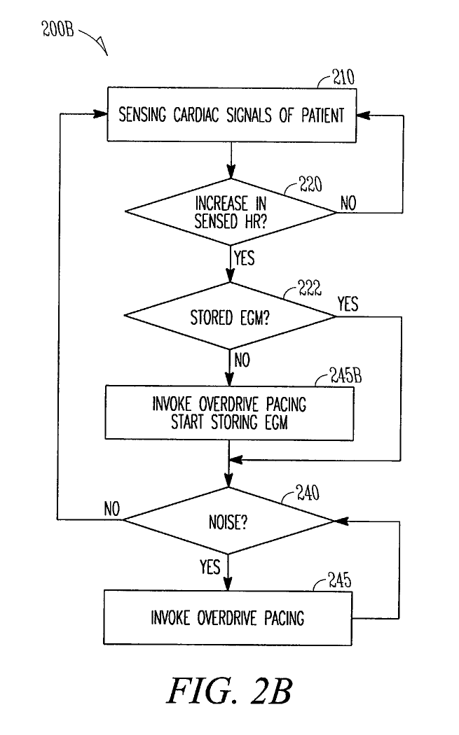

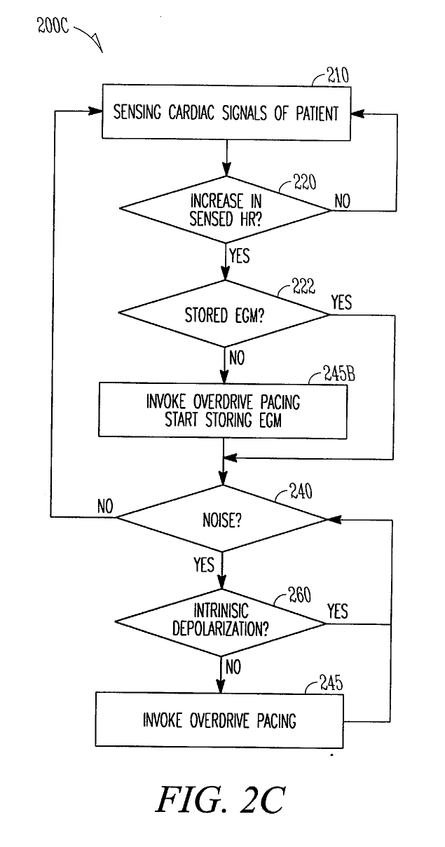

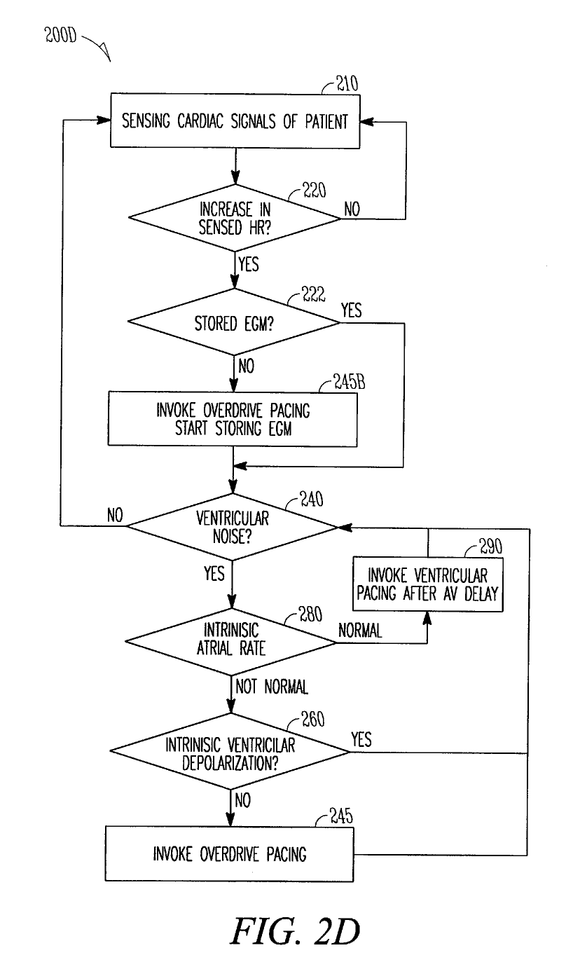

Consequently, in certain examples, an implantable medical device such as the device 100 illustrated in FIG. 1 executes a technique to pace the heart at a normal (or low rate limit) rate, or to automatically overdrive the sensed intrinsic heart rate, upon detection of an indication of an increased sensed heart rate in a cardiac signal. The device 100 may also invoke a combination of normal and overdrive pacing. In another example, the device 100 paces at either a normal rate or an overdrive rate as a function of an indication of the presence of noise in that cardiac signal. This overdriving includes generating pacing pulses at a rate that is higher than the rate of the patient so as to override the estimated intrinsic heart rate of the patient. The overdrive pacing helps to prevent competitive pacing, and helps to avoid pacing during the vulnerable period of the myocardium. Examples of this heart rate sensing, noise sensing, and pacing technique are illustrated in FIGS. 2A-2D.

An estimate of a patient's intrinsic rate is needed to determine the normal pacing rate and the overdrive pacing rate. One way this may be obtained is by continuously monitoring and recording a history of the patient's heart rate. When a heart rate increase is detected with an indication of the presence of noise in the cardiac signal, this history is evaluated to determine the heart rate a short period of time before the onset of the heart rate increase. For example, the heart rate present about five seconds before the onset of the heart rate increase may be used. Alternatively, the heart rate present at a predetermined number of beats before the onset of the heart rate increase may be used (e.g., about eight beats before the onset of the heart rate increase). Another manner to estimate a patient's intrinsic rate may involve the use of a physiologic rate sensor (e.g., based on minute ventilation or an accelerometer) to provide an estimate of the patient's intrinsic heart rate.

FIG. 2A illustrates an example of a process 200A to determine if an increase in a patient's sensed heart rate is the result of noise, and the actions that are taken based on that determination. The process 200A is particularly suited to patient with indications of pacer dependency. In FIG. 2A, a heart rate detector circuit such as heart rate detector circuit 120 in FIG. 1 senses cardiac signals of a patient at 210. If an increase in the heart rate is sensed at 220, overdrive pacing at a rate that is greater than the estimated intrinsic rate of the patient is invoked at 245. The cardiac signal is then analyzed at 240 to determine if there is noise in the cardiac signal. If noise is identified in the cardiac signal at 240, overdrive pacing is continued at 245. In a particular example, the overdrive pacing is at a rate of approximately 120% of the estimated intrinsic rate of the patient. By pacing at 120% of the patient's estimated intrinsic rate, the pacing rate will override the patient's estimated intrinsic rate. In this example, pacing is invoked at this point because the sensed increase of the heart rate may not be an actual increase in the heart rate, but rather may result from the presence of noise in the cardiac signal. If sensed as actual heart beats, this noise may improperly inhibit delivery of pacing pulses when such pacing pulses are actually needed. If noise is not identified in the cardiac signal at 240, the process 200A reverts to sensing and monitoring the cardiac signals of the patient at 210.

In an example, a sensed heart rate is considered to be an increased sensed heart rate when the duration between two or more consecutive heart beats is 10% less than that of a running average (also referred to as the patient's intrinsic rate) of the duration between heart beats for that patient. A sudden rate increase is typically represented by a specified number of consecutive accelerated beats. The number can be specified by being a hard fixed number or by being a programmable number programmed with a device programmer. As an illustrative example, a sudden rate increase is defined as three consecutive accelerated beats. In another example, a sudden rate increase is defined as six consecutive accelerated beats. An accelerated beat is declared by some criterion other than a comparison to a fixed tachyarrhythmia rate threshold (i.e., without a comparison to one or more tachyarrhythmia heart rate zones or one or more tachyarrhythmia detection cutoff heart rates). In other examples, an accelerated beat is identified when a difference between a last average ventricular contraction interval (V-V interval) and the current V-V interval is greater than a specified percentage of the last average V-V interval. As an illustrative example, an accelerated beat is identified when the difference between the last average V-V interval and the current V-V interval is greater than ten percent (10%) of the last average V-V interval, i.e.,

VV_{AVG}(n−1)−VV(n)>(0.1)*(VV_{AVG}(n−1)),

where VV_{AVG}(n−1) is the last or previous average V-V interval and VV(n) is the current V-V interval. If a current beat is not an accelerated beat, the current V-V interval will be close to the average interval and the difference will be close to zero. If the current beat is an accelerated beat, the current V-V interval will be smaller than the average interval value and the difference will increase to a quantity larger than zero. As the current V-V interval decreases, eventually the difference will exceed the specified percentage difference and the beat is identified as an accelerated beat.

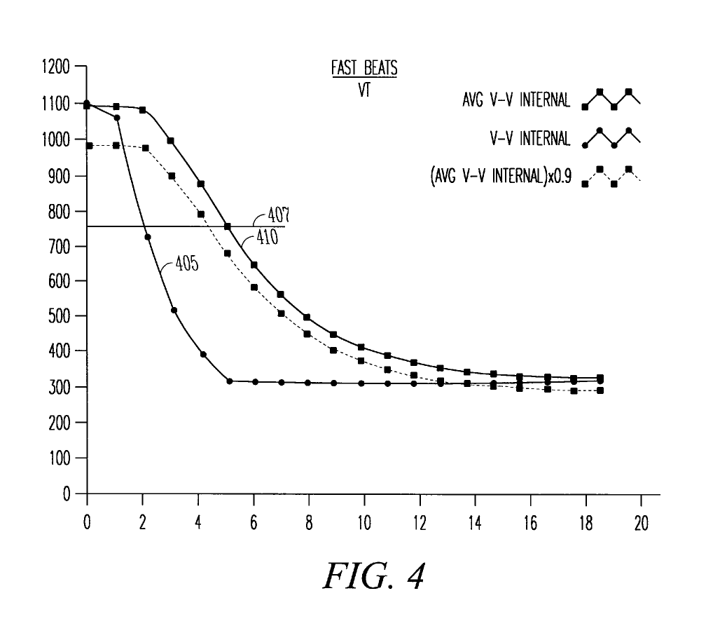

There may be a complication in calculating accelerated beats. If the accelerated beat intervals are included in the calculation of the average V-V interval, the accelerated beats will skew the average to faster intervals or a fast interval steady state if the sudden rate increase is sustained. This is illustrated in FIG. 4. An onset of accelerated beats that are sustained is shown by graph 405. Graph 410 show the effect on the average V-V interval. It can be seen in the graphs that the fast V-V intervals and the average V-V interval converge, making it difficult for subsequent accelerated beats to be detected. In some examples, this complication is overcome by the controller circuit 110 not including the accelerated beats in the calculation of the average V-V interval. In another example, any accelerated beat that occurs after a specified number of accelerated beats, such as three accelerated beats for example, is not used to update the average V-V interval.

In another example, the controller circuit 110 calculates a temporary average V-V interval, different from the last average V-V interval, using the number of consecutive accelerated beats until a sudden rate increase is defined. The temporary average V-V interval is not updated like the normal average V-V interval and is used to identify an accelerated beat after the average V-V interval converges to fast V-V intervals. For example, when an initial sudden rate increase is declared, such as after detecting a third consecutive accelerated beat, the average V-V interval is updated but becomes a temporary average V-V interval as illustrated in FIG. 4 at line 407. The normal average V-V interval is preserved. Any V-V interval shorter than the temporary V-V interval is deemed to be an accelerated beat.

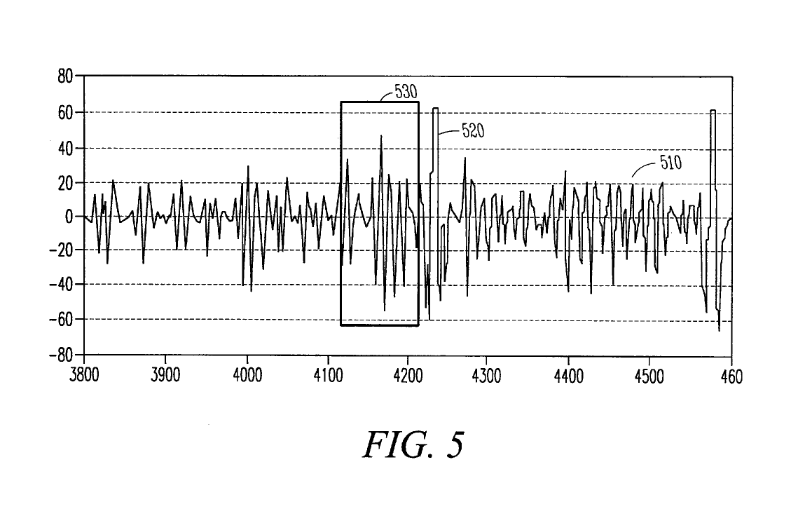

In an example, the evaluation of the noise level in the cardiac signal at 240 may be performed with a noise sensing circuit such as the noise sensing circuit 130 illustrated in FIG. 1. The noise sensing circuit can examine a cardiac signal of approximately two seconds in duration. A cardiac signal of approximately two seconds in duration assures that an intrinsic depolarization will be present in that signal. A potential intrinsic heart beat is then identified in that window by locating the largest peak. As explained in further detail below, an intrinsic beat can be confirmed by examining the amplitude, width, or morphology of the signal. The noise sensing circuit then opens up a 200 ms or similar noise window before the intrinsic heart beat, and examines the cardiac signal in that window for the presence of noise. FIG. 5 illustrates an example of a cardiac signal 510, an intrinsic heart beat 520, and a noise window 530 located before the intrinsic heart beat 520. Since no intrinsic depolarization would be expected during this noise window that immediately precedes an intrinsic depolarization, the cardiac signal during the noise window may be used to identify noise by identifying the largest amplitude peak in the noise window and comparing it to a threshold value. If the largest amplitude peak exceeds the threshold value, then the beat is deemed noisy. Optionally, the cardiac signal in the noise-window may be lowpass filtered, and if the largest amplitude peak in the noise window changes as a result of such lowpass filtering, such change may be used to identify the presence of spike noise associated with electromagnetic interference (EMI). Other methods of noise detection may additionally or alternatively be used. One such method includes summing absolute values (or squaring) of the cardiac signal segment during the noise window, and if the result exceeds a threshold value, deeming the beat to be noisy. Another such method includes counting zero-crossings and if such count exceeds a threshold value, then deeming the beat to be noisy. In one example, that noise window begins approximately 200 milliseconds before the intrinsic heart beat, and it has a duration of approximately 150 milliseconds. If it is determined at 240 that there is noise in this window, then the system invokes overdrive pacing at 245. If noise is not present, then the system continues to examine the cardiac signal for noise at 240, and if no noise is identified at 240 within two seconds or so, the process 200A reverts back to sensing cardiac signals at 210.

FIG. 2B illustrates an example of an alternative process 200B to invoke overdrive pacing upon detecting an increase in the sensed heart beat and the presence of noise in a cardiac signal. The process 200B is suited to a patient who does not have indications of pacer dependency. At 210, a patient's heart rate is monitored. If a sensed increase in the heart rate is detected at 220, the process 200B determines if there are stored cardiac signals in the device at 222. If there are stored cardiac signals, the process 200B checks for noise at 240. If there are no stored cardiac signals, overdrive pacing at a rate greater than the estimated intrinsic rate of the patient is invoked at 245B. In this example, at 245B, the process 200B begins to store the cardiac signals in a memory. The process 200B then analyzes the stored cardiac signals at 240 to determine if any noise is present in the stored cardiac signal. If noise is present in the stored signal, overdrive pacing is continued at 245. If no noise is found in the cardiac signal, the process 200B reverts to sensing and monitoring cardiac signals at 210.

FIG. 2C illustrates another example of a process 200C to initiate overdrive pacing of a patient upon detecting an increase in a patient's sensed heart rate and the presence of noise in a cardiac signal. At 260, the process 200C in FIG. 2C determines whether there is an intrinsic depolarization in the cardiac signal. The depolarization confirmation circuit 155 may be used to determine whether a candidate depolarization (e.g., largest peak during a given time window) is an actual intrinsic depolarization. The depolarization confirmation circuit 155 first determines if the noise level is relatively low. In certain examples, the noise level is deemed low if the largest peaks in the noise window (e.g., as discussed above) are less than ⅓ the amplitude of the candidate QRS peak in the cardiac signal. The depolarization confirmation circuit can additionally or alternatively compare one or more of the candidate peak amplitude, morphology, or width to similar values from a normal sinus rhythm (NSR) beat, with similarity tending to indicate that the candidate beat is an actual intrinsic depolarization, and dissimilarity tending to indicate that the candidate intrinsic depolarization is not an actual depolarization, but instead represents noise. At 260, if there is no intrinsic depolarization, the process 200C continues to overdrive pace the patient at 245. If an intrinsic depolarization is confirmed in the cardiac signal by the depolarization confirmation circuit 155 at 260, the process 200C continues to monitor for noise at 240.

FIG. 2D illustrates another example of a process 200D to determine whether overdrive pacing should be invoked based on detecting an increase in a sensed heart rate and the presence of noise in a cardiac signal. The process 200D is particularly applicable to devices that pace one or both ventricular chambers based on the signal in the atrial channel. In such devices, to determine if the quality of the signal in the atrial channel is of sufficient quality (given that there is evidence that there is noise on the ventricular channel) to use for determining the timing for pacing one or both ventricular chambers, the device can either determine the atrial rate is normal, or determine if there is no sudden change in the atrial rate or amplitude during the timeframe of the onset of noise in the ventricular channel. In process 200D, it is determined at 280 whether the estimated intrinsic heart rate is within a specified range (i.e., the atrial rate is normal). If it is not, the process 200D analyzes the cardiac signal for an intrinsic ventricular depolarization at 260 as in process 200C of FIG. 2C. At 280, if the atrial heart rate detector circuit 180 determines that the intrinsic atrial heart signal is usable for basing ventricular timing then, at 290, a ventricular pace can be delivered after an AV delay, and the cardiac signals of the patient are monitored for the continued presence of noise at 240. If no ventricular depolarization is detected at 260, or it is uncertain if there is a ventricular depolarization, overdrive pacing is continued at 245.

In an example, invoking overdrive pacing based on detecting noise in an implantable medical device system by one or more of the processes outlined in FIGS. 2A-2D may be implemented as an ON/OFF feature. It can be activated when other noise detection algorithms fail to detect the presence of noise, function as a backup check to determine if there is noise in the system, and invoke overdrive pacing when such pacing is needed.

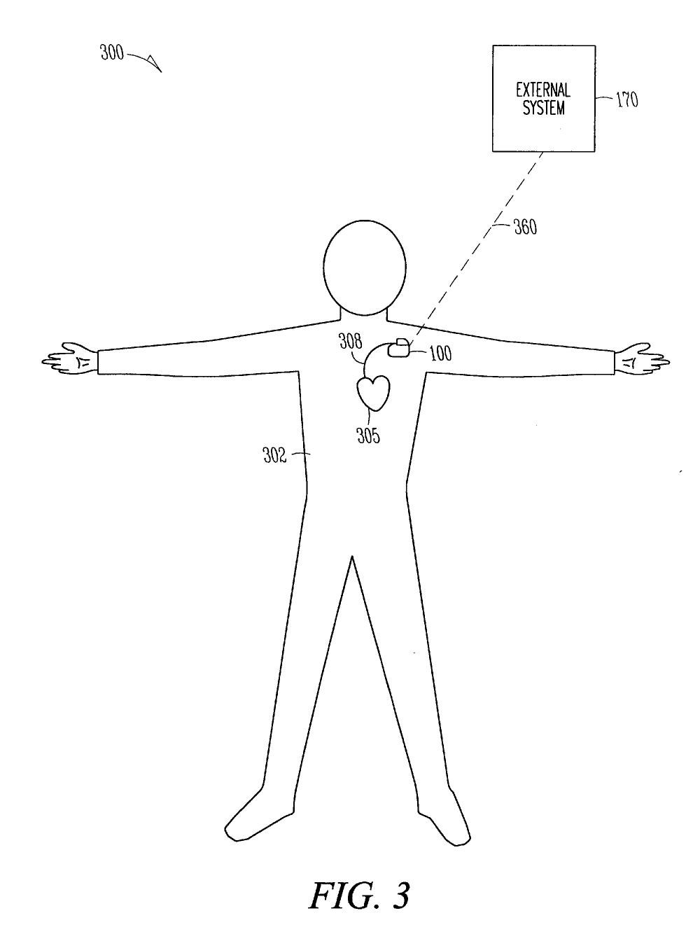

FIG. 3 is a block diagram illustrating an example of a medical device system 300, and portions of an environment in which it is used. In this environment, the environment includes a body 302 with a heart 305. System 300 includes an implantable medical device 100, a lead system 308, an adjunct device or system 170, and a wireless telemetry link 360. Heart rate data, pacing data, EGM data, and other data may be transferred from the device 100 to the external system 170 via the telemetry link 360. The telemetered data loaded into the device 170 can then be used for analysis and interpretation either immediately or at a later time. The ON/OFF feature disclosed in the previous paragraph may be invoked by a physician or other health care provider via the telemetry link 360.

The relationship between a rate and an interval, as used in this document, is the relationship between a frequency and its corresponding period. If a rate is given in beats per minute (bpm), its corresponding interval in milliseconds is calculated by dividing 60,000 by the rate (where 60,000 is the number of milliseconds in a minute). Any process, such as a comparison, using the rates is to be modified accordingly when the intervals are used instead. For example, if a tachyarrhythmia is detected when the ventricular rate exceeds a tachyarrhythmia threshold rate, an equivalent process is to detect the tachyarrhythmia when the ventricular interval falls below a tachyarrhythmia threshold interval. The appended claims should be construed to cover such variations. For example, atrial and ventricular intervals should be construed as equivalent to the atrial and ventricular rates, respectively.

In the foregoing detailed description of embodiments of the invention, various features are grouped together in one or more embodiments for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the detailed description of embodiments of the invention, with each claim standing on its own as a separate embodiment. It is understood that the above description is intended to be illustrative, and not restrictive. It is intended to cover all alternatives, modifications and equivalents as may be included within the scope of the invention as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

As used in this disclosure, the term “circuit” is broadly meant to refer to hardware, software, and a combination of hardware and software. That is, a particular function may be implemented in specialized circuits, in software executing on general processor circuits, and/or a combination of specialized circuits, generalized circuits, and software.

The abstract is provided to comply with 37 C.F.R. 1.72(b) to allow a reader to quickly ascertain the nature and gist of the technical disclosure. The Abstract is submitted with the understanding that it will not be used to interpret or limit the scope or meaning of the claims.

What is claimed is:

1. A system comprising an implantable medical device, the medical device comprising:
a controller circuit;
a heart rate detector circuit coupled to the controller circuit, the heart rate detector circuit for detecting an increase in heart rate in a cardiac signal;
a pacing circuit coupled to the controller circuit; and
a noise sensing circuit coupled to the controller circuit;
wherein the pacing circuit generates pulses when the heart rate detector circuit senses an increase in the heart rate in the cardiac signal and the noise sensing circuit senses a level of noise in the cardiac signal that is greater than a specified amount.

2. The system of claim 1, wherein the pulses generated by the pacing circuit are at a rate greater than an intrinsic rate of a patient to override the intrinsic rate of the patient.

3. The system of claim 1, wherein the implantable medical device is a demand pacer.

4. The system of claim 1, wherein the pacing circuit discontinues generating pulses when the noise sensing circuit senses a level of noise that subsides below a specified amount.

5. The system of claim 1, wherein the system is configured so that first the pacing circuit initiates pacing to the patient in response to the detected increase in heart rate by the heart rate sensing circuit, and second the noise sensing circuit then determines whether the noise in the cardiac signal exceeds the specified amount.

6. The system of claim 1, wherein the system is configured so that first the noise sensing circuit determines whether the noise in the cardiac signal exceeds the specified amount in response to the detected increase in heart rate by the heart rate sensing circuit, and second the pacing circuit then paces the patient when the noise in the cardiac signal exceeds the specified amount.

7. The system of claim 6, further comprising a memory circuit to store the cardiac signal, and wherein the noise sensing circuit analyzes the stored cardiac signal to determine the level of noise in the cardiac signal.

8. The system of claim 1, further comprising a depolarization confirmation circuit to determine the presence of an intrinsic depolarization in the cardiac signal, and wherein the pacing circuit continues generating pulses when there is no depolarization confirmed in the cardiac signal; and further wherein the pacing circuit discontinues generating pulses when there is an intrinsic depolarization confirmed in the cardiac signal.

9. The system of claim 8, wherein
the depolarization confirmation circuit is configured to identify an intrinsic heart beat in the cardiac signal;
the noise sensing circuit is configured to establish a noise measurement window before the intrinsic heart beat; and
the noise sensing circuit is further configured to determine whether the cardiac signal in the noise measurement window exceeds a specified amount.

10. The system of claim 1, wherein the heart rate detector circuit detects an increase in heart rate by identifying at least two consecutive sensed heart beats wherein the duration between the two consecutive beats is at least a specified amount less than a measure of a cardiac heart rate for the patient.

11. The system of claim 1, further comprising:
an atrial heart rate detector circuit coupled to the controller, the atrial heart rate detector circuit for determining whether an atrial heart rate is within a specified range for the patient; and
a cardiac resynchronization therapy circuit for generating cardiac resynchronization therapy after an AV delay when the atrial heart rate is within a specified range.

12. The system of claim 1, further comprising:
an atrial heart rate detector circuit coupled to the controller, the atrial heart rate detector circuit for determining whether an atrial heart rate is within a specified range for the patient; and wherein
the pacing circuit generates pacing pulses after an AV delay when the atrial heart rate is within a specified range.

13. A process comprising:
sensing a cardiac signal of a patient using an implantable medical device;
analyzing the cardiac signal and identifying a sensed increase in a heart rate of the patient;
determining whether noise in the cardiac signal exceeds a specified amount when there is an increase in the sensed heart rate; and
pacing the patient when there is an identified increase in the sensed heart rate and the noise in the cardiac signal exceeds the specified amount.

14. The process of claim 13, wherein the pacing the patient is at a rate that is greater than an intrinsic rate of the patient to override the intrinsic rate.

15. The process of claim 13, wherein the identifying an increase in the heart rate of a patient includes comparing the sensed increase in the heart rate of a patient to an intrinsic heart rate of the patient, and further wherein the intrinsic heart rate of the patient is determined by one or more of an analysis of a recorded history of a patient's heart rate and an analysis of an output of a physiologic rate sensor.

16. The process of claim 13, further comprising discontinuing the pacing the patient when the noise in the cardiac signal subsides.

17. The process of claim 13, comprising first initiating the pacing of the patient in response to the identified sensed increase in the heart rate, then performing the determining whether noise in the cardiac signal exceeds the specified amount.

18. The process of claim 13, comprising first determining whether the noise in the cardiac signal exceeds the specified amount in response to the identifying a sensed increase in the heart rate, then performing the pacing of the patient when the noise in the cardiac signal exceeds the specified amount.

19. The process of claim 18, wherein the cardiac signal is stored in the implantable medical device, and wherein the determining whether noise in the cardiac signal exceeds the specified amount comprises analyzing the stored cardiac signal.

20. The process of claim 13, further comprising:
determining whether there is an intrinsic depolarization in the cardiac signal;
maintaining the pacing of the patient when there is no confirmed intrinsic depolarization in the cardiac signal; and
discontinuing the pacing of the patient when there is a confirmed intrinsic depolarization in the cardiac signal.

21. The process of claim 13, further comprising:
determining whether an atrial heart rate is within a specified range for the patient; and
invoking pacing pulses after an AV delay when the atrial heart rate is within the specified range.

22. The process of claim 13, wherein the determination of whether there is noise in the cardiac signal comprises:
identifying an intrinsic heart beat in the cardiac signal;
establishing a noise measurement window before the intrinsic heart beat; and
determining whether the cardiac signal in the noise measurement window exceeds a specified amount.

23. The process of claim 13, wherein the identifying an increase in the sensed heart rate further comprises identifying at least two consecutive sensed heart beats wherein the duration between the two consecutive beats is at least a specified amount less than a threshold.

24. A system comprising an implantable medical device, the medical device comprising:
a controller circuit;
a heart rate detector circuit coupled to the controller circuit, the heart rate detector circuit for detecting an increase in heart rate in a cardiac signal;
a pacing circuit coupled to the controller circuit; and
a noise sensing circuit coupled to the controller circuit;
wherein the pacing circuit generates pulses when the heart rate detector circuit senses an increase in the heart rate in the cardiac signal.

25. The system of claim 24, wherein the implantable medical device comprises a demand pacer, and further wherein the pulses generated by the pacing circuit are at a rate that is greater than an intrinsic rate of a patient to override the intrinsic rate.
